(19) United States
(12) Patent Application Publication
(10) Pub. No.: US 2025/0090486 A1
(43) Pub. Date: Mar. 20, 2025

(54) Methods And Kits For Treating Fever In Children With Combined Ibuprofen And Acetaminophen

(71) Applicant: Mark Robert Zonfrillo, West Greenwich, RI (US)

(72) Inventor: Mark Robert Zonfrillo, West Greenwich, RI (US)

(21) Appl. No.: 18/724,411

(22) PCT Filed: Dec. 28, 2022
(86) PCT No.: PCT/US2022/082453

§ 371 Date: Jun. 26, 2024

Related U.S. Application Data
(60) Provisional application No. 63/435,502, filed on Dec. 27, 2022.
(60) Provisional application No. 63/294,286, filed on Dec. 28, 2021.

(51) Int. Cl. A61K 31/192; A61K 31/167; A61K 45/06

(52) U.S. Cl. CPC A61K 31/192; A61K 31/167; A61K 45/06

(57) ABSTRACT

Dosing methods and regimens and various dosage forms including formulations and kits for treating fever in children are provided. One dosing regimen or method for treating a febrile child comprises administering a dose to the child, the dose comprising a) a predetermined amount of ibuprofen, and b) a predetermined amount of acetaminophen, wherein the predetermined amounts are determined by weight of the child. Different carriers such as liquids, solids, and semi-solids can be used in the formulations. Dosage charts including the amounts of ibuprofen and acetaminophen compounds in the formulation and child weights also are provided.

### CROSS-REFERENCE TO RELATED APPLICATIONS

The application claims the benefit of PCT International Application No. PCT/US2022/028453 filed Dec. 28, 2022, which claims the benefit of U.S. Provisional Patent Application No. 63/294,286 filed Dec. 28, 2021 and U.S. Provisional Patent Application No. 63/435, 502 filed Dec. 27, 2022, the entire disclosures of which are hereby incorporated by reference.

### BACKGROUND OF THE INVENTION

### Field of the Invention

The present invention relates generally to dosing methods and regimens and various dosage forms including formulations and formulation kits for treating fever in children. More particularly, the dosage forms comprise a combination of ibuprofen and acetaminophen in specific amounts.

### Brief Review of Related Art

In recent years, parents have become more concerned about when and how they should treat their children with analgesic and antipyretic compounds to reduce fever. Fever is one of the most common pediatric symptoms (Schmidtt B D, (1984), Pediatrics, 74(5 Pt 2):929-36) and it is one of parents' most common reasons for seeking medical attention for their children, most often at emergency departments. The medical profession refers to this phenomenon as “fever phobia” or the parental misconceptions of fever in children (Doria M et al., (2019), Int J Environ Res Public Health, 16(22):4487 (9 pages)). Lack of knowledge may lead parents to either overtreat a febrile child or to give subtherapeutic doses of antipyretic medications. Consequently, some children are at risk of suffering the toxic effects of these medications, while others are brought frequently to the emergency room or doctor's office because of fever that does not seem to respond to conventional medication.

Children with fever may feel hot to the touch, have sweaty or clammy skin, begin to shiver, exhibit an increased heart rate (known as tachycardia), and breath quickly (known as tachypnea). These symptoms, although relatively harmless in most instances, cause parents to become anxious believing that their child is sicker than they actually are. Fevers in children may be indicative of a more harmful condition and of the fever can provide the pediatrician with valuable information as to the underlying cause of the fever. Acetaminophen a/k/a paracetamol is the most widely used antipyretic in children because of its antipyretic and analgesic effects and because of its relative safety.

Linder et al., (Linder M et al., (1999), Isr Med Assoc J, 1(3):158-60) conducted a study was to assess the level of parental knowledge on antipyretic medication, the source of that knowledge, how and when parents decide to treat their children, and the reported effects of that treatment. Linder et al., determined that 24.1% of the parents gave their febrile children less than the recommended daily dose of acetaminophen. Parents in the study population, like parents in other studies, erred in assuming a household teaspoon holds 5 ml although the correct volume is 3 ml. A second cause of underdosing was parental failure to revise the dosage with the child's increasing age and weight. Inappropriately low doses may lead to unnecessary office or emergency room visits for children brought by concerned parents who think the fever is resistant to treatment.

Recently, it has been suggested that ibuprofen in combination with acetaminophen is more effective at reducing fever in a febrile child. There is, however, no consensus in the medical literature.

For example, Erleqyn-Lajeuness et al. (Arch Dis Child, (2006), Arch Dis Child, 91(5):414-6) described a trial of paracetamol at a dose of 15 mg/kg, a ibuprofen at a dose of 5 mg/kg or a combination of paracetamol and ibuprofen in febrile children aged from six months to eleven years. The group receiving the combination showed greater fever reduction when compared to the groups receiving only ibuprofen or paracetamol, however, the reduction was less than a half degree centigrade which was well within the margin of error. Erleqyn-Lajeuness et al. opined that the half degree centigrade difference was not clinically significant enough to warrant routing use of the combination.

Sarrell E M et al., (Arch Pediatr Adolesc Med, 160(2):197-202) report that treating fever in children with alternating acetaminophen and ibuprofen is more effective than treating fever in children with either drug alone. Sarrell E M et al. did not notice any long-term complications. Sarrell E M et al. does note that all of the study participants visited the physicians for treatment, thus minimizing the risks of under/dosing.

Hay A D et al., (BJM, (2008), 2008 Sep. 2, 337:a1302) conducted a review of three studies comparing use of the combination of paracetamol and ibuprofen with use of each individually in febrile children. Hay A D et al. concluded that there was insufficient evidence from the three trials to demonstrate a clinically important decrease in fever with combination therapy despite the desire for parents and clinicians to consider a therapy of ibuprofen in combination with acetaminophen for children with fever. Hay A D et al. advise of observed disadvantages with the combination therapy, including the potential risk of overdosing and/or underdosing, increased costs, potential adverse effects and potential heightened fever phobia.

Pursell E (Arch Dis Child, (2011), 96(12):1175-9 conducted a systematic review of studies comparing combined treatment with paracetamol and ibuprofen with treatment with only paracetamol or only ibuprofen. According to Pursell, that of the six reviewed studies having useful data to evaluate the three treatments for effectiveness on the reduction of the temperature of the febrile child, evidenced a statistically significant benefit, but only a marginally significant clinical benefit. Pursell recommended that the risk of potential overdose due to an increased fever phobia due to the trepidation of parents to administer not one, but two doses, outweighed the observed marginal benefit.

Wong T (Cochrane Database Syst Rev, (2013), 2013(10):CD009572) completed an intervention review of trials comparing a combination therapy of paracetamol and ibuprofen (either together in one dose or administered in alternating doses) with monotherapies of paracetamol and ibuprofen in children presenting fever. Wong concluded that while some evidence supports a finding that the combination therapy may be more effective at reducing a fever state in a child that the individual monotherapies, there was inconclusive evidence to compare improvements in patient discomfort, as well inconclusive evidence as to the effectiveness of alternating doses as opposed to administering both drugs as one dose.

Although national and international guidelines on the management of childhood and adolescent fever are available, some inadequate practices persist, both from parents and healthcare professionals. The main goal of bringing children's temperature back to normal can lead to the choice of inappropriate drugs or non-necessary combination/alternation of antipyretic treatments, i.e. fever phobia which is caused also by the dissemination of unscientific information and social media. While over-the-counter acetaminophen and ibuprofen are available to treat fever in children, significant confusion about dosing and frequency of administration prevails. As the standard weight-based dosing for acetaminophen and ibuprofen are different (15 mg/kg and 10 mg/kg, respectively) as well as the frequency of administration (every four hours and every six hours, respectively), can lead to increased fever phobia resulting in the possibility of a care giver administering too much or too little of either drug resulting in inadequate treatment or dangerous side effects, respectively.

It is therefore increasingly important that pediatricians continue to provide adequate information to parents in order to assess the onset of signs of a possible condition of the child's discomfort rather than focusing only on temperature. In fact, there is no clear and unambiguous definition of discomfort in literature. Clarifying the extent of the feverish child's discomfort and the tools that could be used to evaluate it would therefore help recommend that antipyretic treatment is appropriate only if fever is associated with discomfort.

There is a need for a dosing regimen that takes advantage of the observed improvement in treating a febrile child with a combination of ibuprofen and acetaminophen while diminishing fever phobia. In addition, there is a need for a system of ensuring the correct dosing for a particular patient and prevent potentially detrimental underdosing or overdosing. The objective of the invention of to take advantage of the benefits of a combination of acetaminophen and ibuprofen for the treatment of fever in children to deliver the maximally effective dose that is safe and effective. The present invention overcomes the problems of fever phobia with healthcare givers such as parents by providing a weight-based dosing system. The instant invention provides a means in which to treat febrile children with combined dosages of acetaminophen and ibuprofen in the safest, but maximal amount, that removes the guesswork and anxiety surrounding dosing and frequency regimens for either medication alone.

### SUMMARY OF THE INVENTION

In general, the invention is directed to treating febrile child patients. Dosing methods and regimens and various dosage forms including formulations and formulation kits for treating fever in children are provided.

In one embodiment, the regimen or method of treating a febrile child, comprises administering a dose to the child, the dose comprising a) a predetermined amount of ibuprofen, and b) a predetermined amount of acetaminophen, wherein the predetermined amounts are determined by weight of the child. A single dosage formulation can contain the predetermined amount of ibuprofen and predetermined amount of acetaminophen. The formulation can contain various carriers such as, for example, liquids, solids, and semi-solids.

For example, a liquid carrier such as, for example, drops, solutions, suspensions, emulsions, elixirs, and combinations thereof can be used in the formulations. In other examples solids and semi-solids such as, for example, hard candies, popsicles, gelatin-based chewable sweets, chewable tablets, gums, dissolving tablets, powders, capsules, lozenges, dissolving films, enemas, suppositories, ointments, creams, patches, gels, microneedles, packets, sachets, intranasal sprays, and combinations thereof can be used.

In other embodiments, a first formulation contains the predetermined amount of ibuprofen, and a second formulation contains the predetermined amount of acetaminophen, and the first and second formulations are administered to the febrile child separately.

The invention also provides various pharmaceutical formulations and dosage charts that contain information on the amounts of ibuprofen compound and acetaminophen compound in the formulation and exemplary weights for febrile children who will be treated. The dosage charts provide the correct dosage of the formulation as based on the weight of the febrile child being treated. Medical kits for treating a febrile child are also provided. In one embodiment, the kit includes: a) a formulation for treating a febrile child, the formulation comprising a predetermined amount of ibuprofen compound and a predetermined amount of acetaminophen compound, and b) a dosage chart, the chart having information on the amount of ibuprofen compound in the first formulation, and the amount of acetaminophen compound in the second pharmaceutical formulation, and exemplary weights for children, wherein the dosages of the first and second pharmaceutical formulations are based on the weight of the febrile child being treated.

### BRIEF DESCRIPTION OF THE DRAWING

The novel features that are characteristic of the present invention are set forth in the appended claims. However, the preferred embodiments of the invention, together with further objects and attendant advantages, are best understood by reference to the following detailed description in connection with the accompanying drawing in which:

FIG. 1 is a flowchart showing one embodiment of the administration of the dosage of the combined ibuprofen and acetaminophen in accordance with the present invention.

### DETAILED DESCRIPTION OF THE INVENTION

The present invention relates generally to dosing methods and regimens and various dosage forms including formulations and formulation kits for treating fever in children. More particularly, the dosage forms comprise a combination of ibuprofen and acetaminophen in specific amounts.

In one embodiment, the present invention is directed to a dosing regimen or method of treating a febrile child, comprising administering a dose to the child, the dose comprising a) a predetermined amount of ibuprofen, and b) a predetermined amount of acetaminophen, wherein the predetermined amounts are determined by weight of the child. The invention also encompasses formulations and formulation fits for treating febrile children.

By the term, “febrile” as used herein, it is meant a patient having a temperature of greater than or equal to 38.0° C. (≥100.4° F.).

By the term, “child” or “pediatric” patient, it is meant a person having an age of at least six (6) months and less than twelve (12) years.

By the term, “dose” or “dosage” as used herein, it is meant any ingestible form of the pharmaceutical formulation of this invention including, but not limited to, solid, semi-solid, and liquid compositions. Preferably, the liquid forms will either be the standard concentration of ibuprofen (100 mg per 5 ml) and acetaminophen (160 mg per 5 ml) or double the standard concentration of ibuprofen (100 mg per 2.5 ml) and acetaminophen (160 mg per 2.5 ml). In accordance with the present invention, these formulations contain specific predetermined amounts of the ibuprofen and acetaminophen as discussed further below. Suitable liquid dosage forms include, for example, drops, solutions, suspensions, emulsions, elixirs, and the like. Solid and semi-solid dosage forms include, for example, tablets, capsules, gelatin-based capsules, powders, sachets, packets and the like.

In one preferred embodiment, a single dosage formulation contains the predetermined amount of ibuprofen and predetermined amount of acetaminophen. A wide variety of carriers can be included in the formulation. In one preferred version, the carrier is a liquid. For example, the liquid can be selected from the group consisting of drops, solutions, suspensions, emulsions, elixirs, and combinations thereof. These dosage forms include drops for ophthalmic administration and intravenous solutions. In another preferred version, the carrier is a solid or semi-solid, For example, the solid or semi-solid carrier can be selected from the group consisting of hard candies, popsicles, gelatin-based chewable sweets, chewable tablets or gums for oral administration; dissolving tablets, powders, capsules of sprinkles (pellets or granules), lozenges or dissolving films for addition to food or retention in the oral cavity; rectal capsules, enemas or suppositories for rectal administration; ointments, creams, patches, gels or microneedles for diffusion through the skin for systemic distribution; packets; sachets; and intranasal sprays for insufflation.

The single formulation containing the predetermined amount of ibuprofen and predetermined amount of acetaminophen can be administered to the febrile child every six (6) hours until the fever decreases.

In one embodiment, the dosage form has a specific portion comprising the ibuprofen compound and a specific portion comprising the acetaminophen compound, whereby such compositions are delivered in an immediate release manner after administering the formulation to the febrile child. In a second embodiment, the dosage form of the formulation has a specific portion comprising the ibuprofen compound and a specific portion comprising the acetaminophen compound, whereby such combined compositions of both compounds are delivered in a sustained release manner after administering the formulation to the febrile child. In a third embodiment, the ibuprofen compound is released in an immediate release manner and the acetaminophen compound is released in a sustained release manner. In yet another embodiment, the ibuprofen compound is released in a sustained release manner and the acetaminophen compound is released in an immediate release manner.

In one embodiment, the dose is administered in a ratio of 10 mg/kg parts ibuprofen to about 15 mg/kg parts acetaminophen. As discussed above, in one example, the carrier can be a liquid, and the concentration of ibuprofen in the liquid is 100 mg per 5 ml; and the concentration of acetaminophen in the liquid is 160 mg per 5 ml. In another example, the concentration of ibuprofen in the liquid is 100 mg per 2.5 ml; and the concentration of acetaminophen in the liquid is 160 mg per 2.5 ml.

As discussed above, in one embodiment, the ibuprofen and acetaminophen compounds can be administered in one dosage form. That is, a single dosage formulation contains the ibuprofen and acetaminophen compounds. The ibuprofen and acetaminophen are combined in one dose. In another embodiment, a dosage of the first compound (ibuprofen or acetaminophen) can be administered, and then a dosage of the second compound (ibuprofen or acetaminophen) can be administered. That is, a first formulation contains the ibuprofen, and a second formulation contains the acetaminophen, and the formulations are administered separately.

The first formulation containing the predetermined amount of ibuprofen and the second formulation containing the predetermined amount of acetaminophen each can be administered to the febrile child every six (6) hours until the fever decreases.

The dosage of ibuprofen and acetaminophen can contain a wide variety of additives to impart specific properties to the formulation. These additives are included in addition to the ibuprofen and acetaminophen. Suitable examples of additives include, but are not limited to, vitamins, oral care agents, flavoring agents, alkaline gastrointestinal agents, antacids, other pharmaceuticals, and mixtures thereof.

The alkaline gastrointestinal agents, antacids can be used to neutralize stomach acid and allow the drug formulation to release and be available for absorption as soon as the drug passes from the duodenum into the small intestine, and they can help increase absorption.

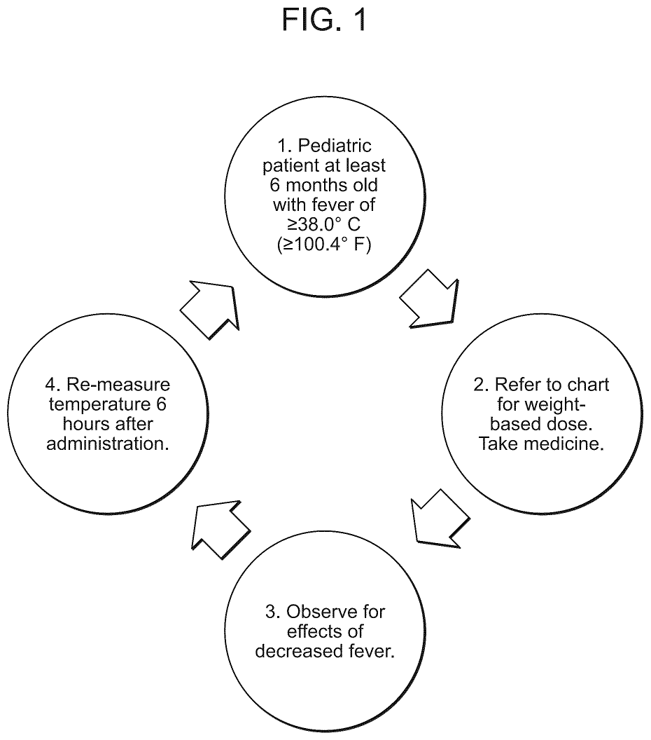

Referring to FIG. 1, a flowchart for administering the dose of a a) a predetermined amount of ibuprofen, and b) a predetermined amount of acetaminophen, wherein the predetermined amounts are determined by weight of the child is shown. In the first step the pediatric patient is examined. In the present invention the dosage forms are intended for pediatric patients having an age of at least six (6) months and a fever of greater than or equal to 38.0° C. (≥100.4° F.). In the second step, the healthcare-giver refers to a dosage chart, which can be provided in a kit as described further below. The dosage chart provides the correct dosage based on the weight of the patient. As described further below, Tables 1, 2, and 3 provide information for different dosage charts. Then, in the third step, the healthcare-giver observes the pediatric patient to determine the effects of the dosage on the patient particularly to see whether the fever has decreased. Next, in the fourth step, the healthcare-giver examines the pediatric patient and takes the patient's temperature. This fourth step is performed about six (6) hours after the first dosage is administered.

The dosing regimens, methods, formulations, and kits of this invention are simple yet effective. The dosing regimens, methods, formulations, and kits of this invention are less confusing for parents and healthcare providers and more cost effective. The present invention minimizes the chances of underdosing, overdosing, and likelihood of serious side effects for the patient.

The invention is illustrated by the following Examples, but these Examples should not be construed as limiting the scope of the invention.

### Example 1

In this Example 1, the dosage for a first pharmaceutical formulation comprising: a) ibuprofen, and b) acetaminophen was determined. The below Table 1 provides the dosage information which can be arranged in a variety of ways so that a health-care provider can easily view it in a user-friendly chart or other display medium. The information in Table 1 contains the correct dosage based on the exact weight of the patient. In particular, referring to Weight Example 1a in Table 1, the exact dosage of the pharmaceutical formulation for a child having a weight of 43.2 kg (95.0 lbs.) is provided. In Weight Example 1a, the dosage contains ibuprofen in the amount of 432 mg and acetaminophen in the amount of 648 mg. The dosage is in the form of an oral liquid, and the amount of liquid containing the active ingredients, (ibuprofen and acetaminophen) is 20.9 milliliters. In another example, Weight Example 3g, the weight of the child is 24.0 kg (52.8 lbs). The dosage contains ibuprofen in the amount of 240 mg and acetaminophen in the amount of 360 mg in 11.6 milliliters of liquid carrier.

TABLE 1
Weight-Based Dosing for Exact Dosing of Combined Ibuprofen and Acetaminophen

| | | | | EXACT DOSING | |
|---|---|---|---|---|---|
| | | | EXACT DOSING | FOR ORAL | |
| | | | FOR ORAL | LIQUID (DROPS, | ORAL LIQUID |
| | | | LIQUID (DROPS, | SOLUTION, | (DROPS, |
| | | | SOLUTION, | SUSPENSION, OR | SOLUTION, |
| | | | SUSPENSION, OR | ELIXIR) [or other | SUSPENSION, OR |
| | | | ELIXIR) [or other | carriers of | ELIXIR) ROUTE - |
| | | | carriers of | administration | EXACT DOSE |
| | | | administration | where an exact | Milliliters |
| | | | where an exact | dose is possible] | administered (exact |
| Weight | Weight | Weight | dose is possible] | acetaminophen | for simultaneous |
| example | (kg) | (lbs) | ibuprofen (mg)* | (mg)* | distribution) * |
| 1a | 43.2 | 95.0 | 432 | 648 | 20.9 |
| 1b | 42.7 | 93.9 | 427 | 640 | 20.7 |
| 1c | 42.1 | 92.7 | 421 | 632 | 20.4 |
| 1d | 41.6 | 91.5 | 416 | 624 | 20.2 |
| 1e | 41.1 | 90.3 | 411 | 616 | 19.9 |
| 1f | 40.5 | 89.2 | 405 | 608 | 19.6 |
| 1g | 40.0 | 88.0 | 400 | 600 | 19.4 |
| 1h | 39.5 | 86.8 | 395 | 592 | 19.1 |
| 1i | 38.9 | 85.7 | 389 | 584 | 18.9 |
| 1j | 38.4 | 84.5 | 384 | 576 | 18.6 |
| 1k | 37.9 | 83.3 | 379 | 568 | 18.4 |
| 1l | 37.3 | 82.1 | 373 | 560 | 18.1 |
| 1m | 36.8 | 81.0 | 368 | 552 | 17.8 |
| 1n | 36.3 | 79.8 | 363 | 544 | 17.6 |
| 1o | 35.7 | 78.6 | 357 | 536 | 17.3 |
| 1p | 35.2 | 77.4 | 352 | 528 | 17.1 |
| 1q | 34.7 | 76.3 | 347 | 520 | 16.8 |
| 1r | 34.1 | 75.1 | 341 | 512 | 16.5 |
| 1s | 33.6 | 73.9 | 336 | 504 | 16.3 |
| 1t | 33.1 | 72.7 | 331 | 496 | 16 |
| 1u | 32.5 | 71.6 | 325 | 488 | 15.8 |
| 2a | 32.0 | 70.4 | 320 | 480 | 15.5 |
| 2b | 31.5 | 69.2 | 315 | 472 | 15.3 |
| 2c | 30.9 | 68.1 | 309 | 464 | 15 |
| 2d | 30.4 | 66.9 | 304 | 456 | 14.7 |
| 2e | 29.9 | 65.7 | 299 | 448 | 14.5 |
| 2f | 29.3 | 64.5 | 293 | 440 | 14.2 |
| 2g | 28.8 | 63.4 | 288 | 432 | 14 |
| 2h | 28.3 | 62.2 | 283 | 424 | 13.7 |
| 2i | 27.7 | 61.0 | 277 | 416 | 13.4 |
| 3a | 27.2 | 59.8 | 272 | 408 | 13.2 |
| 3b | 26.7 | 58.7 | 267 | 400 | 12.9 |
| 3c | 26.1 | 57.5 | 261 | 392 | 12.7 |
| 3d | 25.6 | 56.3 | 256 | 384 | 12.4 |
| 3e | 25.1 | 55.1 | 251 | 376 | 12.2 |
| 3f | 24.5 | 54.0 | 245 | 368 | 11.9 |
| 3g | 24.0 | 52.8 | 240 | 360 | 11.6 |
| 3h | 23.5 | 51.6 | 235 | 352 | 11.4 |
| 3i | 22.9 | 50.5 | 229 | 344 | 11.1 |
| 3j | 22.4 | 49.3 | 224 | 336 | 10.9 |
| 3k | 21.9 | 48.1 | 219 | 328 | 10.6 |
| 4a | 21.3 | 46.9 | 213 | 320 | 10.3 |
| 4b | 20.8 | 45.8 | 208 | 312 | 10.1 |
| 4c | 20.3 | 44.6 | 203 | 304 | 9.8 |
| 4d | 19.7 | 43.4 | 197 | 296 | 9.6 |
| 4e | 19.2 | 42.2 | 192 | 288 | 9.3 |
| 4f | 18.7 | 41.1 | 187 | 280 | 9.1 |
| 4g | 18.1 | 39.9 | 181 | 272 | 8.8 |
| 4h | 17.6 | 38.7 | 176 | 264 | 8.5 |
| 4i | 17.1 | 37.5 | 171 | 256 | 8.3 |
| 4j | 16.5 | 36.4 | 165 | 248 | 8 |
| 4k | 16.0 | 35.2 | 160 | 240 | 7.8 |
| 5a | 15.5 | 34.0 | 155 | 232 | 7.5 |
| 5b | 14.9 | 32.9 | 149 | 224 | 7.2 |
| 5c | 14.4 | 31.7 | 144 | 216 | 7 |
| 5d | 13.9 | 30.5 | 139 | 208 | 6.7 |
| 5e | 13.3 | 29.3 | 133 | 200 | 6.5 |
| 5f | 12.8 | 28.2 | 128 | 192 | 6.2 |
| 5g | 12.3 | 27.0 | 123 | 184 | 6 |
| 5h | 11.7 | 25.8 | 117 | 176 | 5.7 |
| 5i | 11.2 | 24.6 | 112 | 168 | 5.4 |
| 6a | 10.7 | 23.5 | 107 | 160 | 5.2 |
| 6b | 10.1 | 22.3 | 101 | 152 | 4.9 |
| 6c | 9.6 | 21.1 | 96 | 144 | 4.7 |
| 6d | 9.1 | 19.9 | 91 | 136 | 4.4 |
| 6e | 8.5 | 18.8 | 85 | 128 | 4.1 |
| 6f | 8.0 | 17.6 | 80 | 120 | 3.9 |
| 6g | 7.5 | 16.4 | 75 | 112 | 3.6 |
| 6h | 6.9 | 15.3 | 69 | 104 | 3.4 |

*Note—the weight-based dosing in mg is the same as it would be for the individual medications, however the concentration of ibuprofen and acetaminophen is twice that of traditional oral liquid forms (100 mg ibuprofen in each 2.5 ml instead of 100 mg in 5 ml and 160 mg acetaminophen in each 2.5 ml instead of 160 mg in 5 ml) to keep the volume of combined drug similar to that of either individual drug.

### Example 2

In this Example 2, referring to Table 2, information for a second dosage chart was determined. The information in Table 2 contains the correct dosage based on a weight range of the pediatric patient. That is, a key difference between the above-described Table 1 in Example 1 and Table 2 is that the information in Table 1 is based on the exact weight of the pediatric patient, while the information in Table 2 is based on the weight range of the pediatric patient. The dosing in Table 1 can be referred to as “Exact Dosing”, while the dosing in Table 2 can be referred to as “Standard Dosing.”

In particular, referring to Weight Examples 1a to 1u in Table 2, the standard dosing ranges of the pharmaceutical formulation for children having a weight of 43.2 kg (95.0 lbs.) to 32.5 kg (71.6 lbs) is provided. In Weight Examples 1a to 1u, the dosage contains ibuprofen in the amount of 300 mg and acetaminophen in the amount of 480 mg. The dosage is in the form of an oral liquid, and the amount of liquid containing the active ingredients, (ibuprofen and acetaminophen) is 15.0 milliliters. Turning to Weight Examples 3a to 3k in Table 2, the standard dosing ranges of the pharmaceutical formulation for children having a weight of 27.2 kg (59.8 lbs.) to 21.9 kg (48.1 lbs) is provided. In Weight Examples 3a to 3k, the dosage contains ibuprofen in the amount of 200 mg and acetaminophen in the amount of 320 mg in 10 milliliters of liquid.

TABLE 2
Weight-Based Dosing for Ranges of Dosing of Combined Ibuprofen and Acetaminophen

| | | | STANDARD DOSING | STANDARD DOSING | | |
|---|---|---|---|---|---|---|
| | | | RANGES FOR ORAL | RANGES FOR ORAL | For oral liquid | |
| | | | LIQUID (DROPS, | LIQUID (DROPS, | only (DROPS, | Units |
| | | | SOLUTION, | SOLUTION, | SOLUTION, | administered |
| | | | SUSPENSION, | SUSPENSION, | SUSPENSION, | (for other |
| | | | OR ELIXIR) | OR ELIXIR) | OR ELIXIR): | carriers/ |
| | | | [or other carriers | [or other carriers | milliliters | modes of |
| | | | where dosing ranges | where dosing ranges | administered | delivery |
| | | | would apply] | would apply] | (rounded for | where dosing |
| Weight | weight | Wt. | ibuprofen | acetaminophen | standard | ranges would |
| example | (kg) | (lbs) | (mg)* | (mg)* | weight ranges) * | apply) |
| 1a | 43.2 | 95.0 | 300 | 480 | 15 | 3 |
| 1b | 42.7 | 93.9 | 300 | 480 | 15 | 3 |
| 1c | 42.1 | 92.7 | 300 | 480 | 15 | 3 |
| 1d | 41.6 | 91.5 | 300 | 480 | 15 | 3 |
| 1e | 41.1 | 90.3 | 300 | 480 | 15 | 3 |
| 1f | 40.5 | 89.2 | 300 | 480 | 15 | 3 |
| 1g | 40.0 | 88.0 | 300 | 480 | 15 | 3 |
| 1h | 39.5 | 86.8 | 300 | 480 | 15 | 3 |
| 1i | 38.9 | 85.7 | 300 | 480 | 15 | 3 |
| 1j | 38.4 | 84.5 | 300 | 480 | 15 | 3 |
| 1k | 37.9 | 83.3 | 300 | 480 | 15 | 3 |
| 1l | 37.3 | 82.1 | 300 | 480 | 15 | 3 |
| 1m | 36.8 | 81.0 | 300 | 480 | 15 | 3 |
| 1n | 36.3 | 79.8 | 300 | 480 | 15 | 3 |
| 1o | 35.7 | 78.6 | 300 | 480 | 15 | 3 |
| 1p | 35.2 | 77.4 | 300 | 480 | 15 | 3 |
| 1q | 34.7 | 76.3 | 300 | 480 | 15 | 3 |
| 1r | 34.1 | 75.1 | 300 | 480 | 15 | 3 |
| 1s | 33.6 | 73.9 | 300 | 480 | 15 | 3 |
| 1t | 33.1 | 72.7 | 300 | 480 | 15 | 3 |
| 1u | 32.5 | 71.6 | 300 | 480 | 15 | 3 |
| 2a | 32.0 | 70.4 | 250 | 400 | 12.5 | 2.5 |
| 2b | 31.5 | 69.2 | 250 | 400 | 12.5 | 2.5 |
| 2c | 30.9 | 68.1 | 250 | 400 | 12.5 | 2.5 |
| 2d | 30.4 | 66.9 | 250 | 400 | 12.5 | 2.5 |
| 2e | 29.9 | 65.7 | 250 | 400 | 12.5 | 2.5 |
| 2f | 29.3 | 64.5 | 250 | 400 | 12.5 | 2.5 |
| 2g | 28.8 | 63.4 | 250 | 400 | 12.5 | 2.5 |
| 2h | 28.3 | 62.2 | 250 | 400 | 12.5 | 2.5 |
| 2i | 27.7 | 61.0 | 250 | 400 | 12.5 | 2.5 |
| 3a | 27.2 | 59.8 | 200 | 320 | 10 | 2 |
| 3b | 26.7 | 58.7 | 200 | 320 | 10 | 2 |
| 3c | 26.1 | 57.5 | 200 | 320 | 10 | 2 |
| 3d | 25.6 | 56.3 | 200 | 320 | 10 | 2 |
| 3e | 25.1 | 55.1 | 200 | 320 | 10 | 2 |
| 3f | 24.5 | 54.0 | 200 | 320 | 10 | 2 |
| 3g | 24.0 | 52.8 | 200 | 320 | 10 | 2 |
| 3h | 23.5 | 51.6 | 200 | 320 | 10 | 2 |
| 3i | 22.9 | 50.5 | 200 | 320 | 10 | 2 |
| 3j | 22.4 | 49.3 | 200 | 320 | 10 | 2 |
| 3k | 21.9 | 48.1 | 200 | 320 | 10 | 2 |
| 4a | 21.3 | 46.9 | 150 | 240 | 7.5 | 1.5 |
| 4b | 20.8 | 45.8 | 150 | 240 | 7.5 | 1.5 |
| 4c | 20.3 | 44.6 | 150 | 240 | 7.5 | 1.5 |
| 4d | 19.7 | 43.4 | 150 | 240 | 7.5 | 1.5 |
| 4e | 19.2 | 42.2 | 150 | 240 | 7.5 | 1.5 |
| 4f | 18.7 | 41.1 | 150 | 240 | 7.5 | 1.5 |
| 4g | 18.1 | 39.9 | 150 | 240 | 7.5 | 1.5 |
| 4h | 17.6 | 38.7 | 150 | 240 | 7.5 | 1.5 |
| 4i | 17.1 | 37.5 | 150 | 240 | 7.5 | 1.5 |
| 4j | 16.5 | 36.4 | 150 | 240 | 7.5 | 1.5 |
| 4k | 16.0 | 35.2 | 150 | 240 | 7.5 | 1.5 |
| 5a | 15.5 | 34.0 | 100 | 160 | 5 | 1 |
| 5b | 14.9 | 32.9 | 100 | 160 | 5 | 1 |
| 5c | 14.4 | 31.7 | 100 | 160 | 5 | 1 |
| 5d | 13.9 | 30.5 | 100 | 160 | 5 | 1 |
| 5e | 13.3 | 29.3 | 100 | 160 | 5 | 1 |
| 5f | 12.8 | 28.2 | 100 | 160 | 5 | 1 |
| 5g | 12.3 | 27.0 | 100 | 160 | 5 | 1 |
| 5h | 11.7 | 25.8 | 100 | 160 | 5 | 1 |
| 5i | 11.2 | 24.6 | 100 | 160 | 5 | 1 |
| 6a | 10.7 | 23.5 | 50 | 80 | 2.5 | 0.5 |
| 6b | 10.1 | 22.3 | 50 | 80 | 2.5 | 0.5 |
| 6c | 9.6 | 21.1 | 50 | 80 | 2.5 | 0.5 |
| 6d | 9.1 | 19.9 | 50 | 80 | 2.5 | 0.5 |
| 6e | 8.5 | 18.8 | 50 | 80 | 2.5 | 0.5 |
| 6f | 8.0 | 17.6 | 50 | 80 | 2.5 | 0.5 |
| 6g | 7.5 | 16.4 | 50 | 80 | 2.5 | 0.5 |
| 6h | 6.9 | 15.3 | 50 | 80 | 2.5 | 0.5 |

* Note -
the weight-based dosing in mg is the same as it would be for the individual medications, however the concentration of ibuprofen and acetaminophen is twice that of traditional oral liquid forms (100 mg ibuprofen in each 2.5 ml instead of 100 mg in 5 ml and 160 mg acetaminophen in each 2.5 ml instead of 160 mg in 5 ml) to keep the volume of combined drug to that of either individual drug.

### Example 3

In this Example 3, information for a third dosage chart was determined as shown in Table 3. The information in Table 3 contains the correct dosage based on a weight range of the pediatric patient. Note that the pound to kilogram conversions are not exact, and are rounded to whole number ranges for ease of use. The information in Table 3 incorporates the information contained in above-described Table 2. In particular, the Weight Examples 1a to 1u; 2a to 2i; 3a to 3k; 4a to 4k; 5a to 5i; and 6a to 6h from Table 2 is included in Table 3. In addition, Table 3 includes age information for the pediatric patient. While age can be used as a reference, the weight ranges should primarily guide the dose to administer.

TABLE 3
Abbreviated, Rounded, Standard Weight- and Age-Based Ranges
for Dosing of Combined Ibuprofen and Acetaminophen

| | | | | | | | Dose (units | | |
|---|---|---|---|---|---|---|---|---|---|
| | | | | | | | of other | | |
| | | | | | | | carriers | | |
| | | | | | | | including | ibuprofen | acetaminophen |
| Weight | Weight | Weight | Age | | Dose | | chewable, | amount | amount |
| examples | (lbs)* | (kg)* | (years) | | (milliliters)* | | etc.) | (mg)* | (mg)* |
| 1 (a-u) | 72-95 | 33-43 | 11 | years | 15 | mL | 3 | 300 | 480 |
| 2 (a-i) | 60-71 | 28-32 | 9-10 | years | 12.5 | mL | 2.5 | 250 | 400 |
| 3 (a-k) | 48-59 | 22-27 | 6-8 | years | 10 | mL | 2 | 200 | 320 |
| 4 (a-k) | 36-47 | 17-21 | 4-5 | years | 7.5 | mL | 1.5 | 150 | 240 |
| 5 (a-i) | 24-35 | 11-16 | 2-3 | years | 5 | mL | 1 | 100 | 160 |

| | | | | | | | | |
|---|---|---|---|---|---|---|---|---|
| 6 (a-h) | 15-23 | 6-10 | 6 months-2 years | 2.5 | ml | 0.5 | 50 | 80 |

*Note -
the weight-based dosing in mg is the same as it would be for the individual medications, however the concentration of ibuprofen and acetaminophen is twice that of traditional oral liquid forms (100 mg ibuprofen in each 2.5 ml instead of 100 mg in 5 ml and 160 mg acetaminophen in each 2.5 ml instead of 160 mg in 5 ml) to keep the volume of combined drug to that of either individual drug. Note that the pound to kilogram conversions are not exact, and are rounded to whole number ranges for ease of use.

As described above in Tables 1, 2, and 3, different dosage charts are provided in accordance with the current invention. By the term, “dosage chart” as used herein, it is meant any display medium containing dosage information including, but not limited to, cards, sheets, pads, posters, tables, outlines, tablets, graphics, and the like. The dosage chart can be in electronic or non-electronic form. The dosage charts contain information on the amounts of ibuprofen compound and acetaminophen compound in exemplary pharmaceutical formulation and exemplary weights for febrile children who will be treated. (Tables 1 and 2 include exact child weights and Table 3 includes child weight ranges.) The dosage charts provide the correct dosage of the formulation as based on the weight of the febrile child being treated. In the Examples provided in Tables 1 and 2, the formulation contains a liquid carrier, and the dose is given in terms of milliliters (ml). In the Examples provided in Table 3, some examples are directed to a formulation containing a liquid carrier, and the dose is given in terms of milliliters (ml). Other examples are directed to a formulation containing solid or semi-solid carriers, and the dose is given in terms of measured units.

The dosage charts can be used for treating a child with fever in accordance with the invention. For example, in one method, the care giver can first take the temperature of the child to determine if the fever can be treated. If the fever can be treated, the child is weighed, and then the correct dosage of the pharmaceutical formulation (as provided in above Tables 1, 2, and 3) is delivered to the child. Then, the caregiver takes the temperature of the child after a predetermined amount of time to determine if the child is with a fever that can be treated. If the child still has a fever that can be treated, the correct dosage of the pharmaceutical formulation (as provided in above Tables 1, 2, and 3) is delivered to the child until the child is no longer febrile.

The present invention also includes medical kits for treating a febrile child. In one embodiment, the kit includes: a) a formulation for treating a febrile child, the formulation comprising a predetermined amount of ibuprofen compound and a predetermined amount of acetaminophen compound, and b) a dosage chart, the chart having information on the amount of ibuprofen compound in the first formulation, and the amount of acetaminophen compound in the second pharmaceutical formulation, and exemplary weights for children, wherein the dosages of the first and second pharmaceutical formulations are based on the weight of the febrile child being treated. In another embodiment, the kit includes: a) a first pharmaceutical formulation for treating a febrile child, the first formulation comprising a predetermined amount of ibuprofen compound, b) a second pharmaceutical formulation for treating a febrile child, the first formulation comprising a predetermined amount of acetaminophen compound, and c) the above-described dosage chart. The kits can further include means for measuring the dosage of the formulation (for example, a measuring cup, measuring spoon, vial, tube, syringe, and the like) so that the measured dosage can be delivered to the febrile child. The kits also can have a delivery system wherein the first and second formulations is disposed in containers such as, for example, ampules, vials, or tubes, so that the formulations can be administered simultaneously to the child.

Although the present invention is primarily directed to dosage forms of combined ibuprofen and acetaminophen, it is understood that non-steroidal, anti-inflammatory compounds other than ibuprofen could be used (such as naproxen), and analgesic and antipyretic compounds other than acetaminophen could be used provided the dosage amounts were adjusted accordingly depending upon the specific drugs in the formulation.

For pediatric patients 6 months to 12 years old, the abbreviated, rounded, standard oral doses of ibuprofen (by and in itself) typically range from about 50 mg to about 300 mg (Table 3). For the liquid dosage form (for example, drops, solutions, emulsions, suspensions, or elixirs), this equates to 2.5 ml to 15 ml. The dose of ibuprofen is given no more frequently than every 6 hours. Likewise, for pediatric patients 6 months to 12 years old, the abbreviated, rounded, standard oral doses of acetaminophen (by and in itself) typically range from about 80 mg to about 480 milligrams (Table 3). For the liquid dosage form (for example, drops, solutions, emulsions, suspensions, or elixirs), this equates to 2.5 to 15 milliliters. The dose of acetaminophen is given no more frequently than every 4 hours.

Now, in accordance with the present invention, the concentration of the combined liquid dosage form (for example, drops, solutions, emulsions, suspensions, or elixirs) is double that of the standard dosage. Therefore, the amount of volume used in the combined liquid dosage form of ibuprofen and acetaminophen is identical to (i.e, no more than) the volume used in a standard dosage of ibuprofen when the ibuprofen is given solely. Similarly, the amount of volume used in the combined dosage form of ibuprofen and acetaminophen is identical to (i.e, no more than) the amount of acetaminophen used in a common dosage of acetaminophen when the acetaminophen is given solely. As an example, a child who is 55.1 lbs. (25 kg) would receive an abbreviated, rounded, standard dose of ibuprofen of 200 mg (10 ml). (See Table 3, row 3 (a-k).) That same child would receive an abbreviated, rounded, standard dose of acetaminophen of 320 mg (10 ml). (See Table 3, row 3 (a-k).) Given the concentration of each of the drugs in the combined liquid dosage form (for example, drops, solutions, emulsions, suspensions, or elixirs) is double that of the standard dosing, the final volume of the combined volume is no more than the volume used in a standard dosage of either: ibuprofen 200 mg and acetaminophen 320 mg, in a total volume of 10 ml (versus a total volume of 20 ml if the two individual, abbreviated, rounded, liquid doses were given separately.)

The present invention provides effective dosing regimens, methods, formulations, and kits for effectively treating pediatric patients with ibuprofen and acetaminophen in a safe and therapeutic manner. It should be understood the compounds, compositions, formulations, regimens, methods, kits, and the like described and illustrated herein represent only some embodiments of the invention. It is appreciated by those skilled in the art that various changes and additions can be made to the compounds, compositions, formulations, regimens, methods, kits, and the like without departing from the spirit and scope of this invention. It is intended that all such embodiments be covered by the appended claims.

1. A dosing regimen for the treatment of a febrile child, comprising administering a dose to the child, the dose comprising a predetermined amount of ibuprofen compound and a predetermined amount of acetaminophen compound; wherein the predetermined amount of each compound is determined by the weight of the child.

2. The dosing regimen according to claim 1, wherein a single dosage formulation contains the predetermined amount of ibuprofen and predetermined amount of acetaminophen.

3. The dosing regimen according to claim 2, wherein the formulation contains a carrier for the predetermined amount of ibuprofen and predetermined amount of acetaminophen.

4. The dosing regimen according to claim 3, wherein the carrier is a liquid.

5. The dosing regimen according to claim 4, wherein the liquid is selected from the group consisting of drops, solutions, suspensions, emulsions, elixirs, and combinations thereof.

6. The dosing regimen according to claim 3, wherein the carrier is a solid or semi-solid composition.

7. The dosing regimen according to claim 6, wherein the carrier is selected from the group consisting of hard candies, popsicles, gelatin-based chewable sweets, chewable tablets, gums, dissolving tablets, powders, capsules, lozenges, dissolving films, enemas, suppositories, ointments, creams, patches, gels, microneedles, packets, sachets, intranasal sprays, and combinations thereof.

8. The dosing regimen according to claim 2, wherein the formulation containing the predetermined amount of ibuprofen and predetermined amount of acetaminophen is administered to the febrile child every six hours until the fever decreases.

9. The dosing regimen according to claim 4, wherein the carrier is a liquid and the concentration of ibuprofen in the liquid is 100 mg per 5 ml; and the concentration of acetaminophen in the liquid is 160 mg per 5 ml.

10. The dosing regimen according to claim 2, wherein the carrier is a liquid and the concentration of ibuprofen in the liquid is 100 mg per 2.5 ml; and the concentration of acetaminophen in the liquid is 160 mg per 2.5 ml.

11. The dosing regimen according to claim 2, wherein the formulation further comprises an additive selected from the group consisting of vitamins, oral care agents, flavoring agents, alkaline gastrointestinal agents, antacids, other pharmaceuticals, and combinations thereof.

12. The dosing regimen according to claim 11, wherein the additive is an alkaline gastrointestinal agent or antacid.

13. The dosing regimen according to claim 2, wherein the ibuprofen compound and acetaminophen compound are released in an immediate release manner after administering the formulation to the febrile child.

14. The dosing regimen according to claim 2, wherein the ibuprofen compound and acetaminophen compound are released in a sustained release manner after administering the formulation to the febrile child.

15. The dosing regimen according to claim 2, wherein the ibuprofen compound is released in an immediate release manner and the acetaminophen compound is released in a sustained release manner after administering the formulation to the febrile child.

16. The dosing regimen according to claim 2, wherein the ibuprofen compound is released in a sustained release manner and the acetaminophen compound is released in an immediate release manner after administering the formulation to the febrile child.

17. The dosing regimen according to claim 1, wherein a first formulation contains the predetermined amount of ibuprofen and a second formulation contains the predetermined amount of acetaminophen, and the first and second formulations are administered to the febrile child separately.

18. The dosing regimen according to claim 17, wherein the first and second formulations are administered to the febrile child every six hours.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A medical kit for treating a febrile child, comprising
a pharmaceutical formulation for treating a febrile child, the formulation comprising a predetermined amount of ibuprofen compound and a predetermined amount of acetaminophen compound, and
a dosage chart, the chart having information on the amount of ibuprofen compound and amount of acetaminophen compound in the pharmaceutical formulation, and exemplary weights for children, wherein the dosage of the pharmaceutical formulation is based on the weight of the febrile child being treated.

33. The medical kit of claim 32, wherein the kit further comprises a means for measuring the dosage of the pharmaceutical formulation so that the measured dosage can be administered to the febrile child.

34. (canceled)

35. (canceled)

36. (canceled)
